(19) United States
(12) Patent Application Publication
(10) Pub. No.: US 2026/0241060 A1
(43) Pub. Date: Aug. 20, 2026

(54) Probiotic And Cellular Graft Compositions For Uses In Tissue Rebuilding

(71) Applicants: Emory University, Atlanta, GA (US); Children's Healthcare of Atlanta, Inc., Atlanta, GA (US)

(72) Inventors: Steven L. Goudy, Atlanta, GA (US); Rheinallt M. Jones, Atlanta, GA (US); Andre Burnham, Atlanta, GA (US); Keerthi Chinniampalayam, Atlanta, GA (US); Monica Behara, Cambridge, MA (US)

(21) Appl. No.: 19/162,380

(22) PCT Filed: Feb. 27, 2024
(86) PCT No.: PCT/US2024/017408

§ 371 Date: Sep. 4, 2025

Related U.S. Application Data
(60) Provisional application No. 63/452,083, filed on Mar. 14, 2023.
(60) Provisional application No. 63/452,088, filed on Mar. 14, 2023.

(51) Int. Cl. A61L 24/00

(52) U.S. Cl. CPC A61L 24/0073; A61L 24/0005; A61L 24/0015; A61L 2300/412; A61L 2300/64; A61L 2430/36

(57) ABSTRACT

This disclosure reports methods of improving tissue wound healing or growing tissues by using biodegradable graft compositions optionally comprising an exogenously added beneficial bacteria and optionally comprising exogenously added isolated macrophages or other cells. In certain embodiments, this disclosure relates to surgical methods of improving the healing of oral issues after a surgical intervention by implanting graft compositions reported herein on, in, or around the surgical site.

### CROSS-REFERENCE TO RELATED APPLICATIONS

This application claims the benefit of U.S. Provisional Application No. 63/452,083 filed Mar. 14, 2023 and U.S. Provisional Application No. 63/452,088 filed Mar. 14, 2023. The entirety of each of these applications is hereby incorporated by reference for all purposes.

### STATEMENT REGARDING FEDERALLY SPONSORED RESEARCH OR DEVELOPMENT

This invention was made with government support under DE028905 and DE030632 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 23110PCT.xml. The XML file is 3 KB, was created on Feb. 27, 2024, and is being submitted electronically via the USPTO patent electronic filing system.

### BACKGROUND

If bones and tissues of the face do not join properly during fetal growth, a cleft palate, cleft lip, or both, can develop. Surgical interventions are commonly used to correct these defects. For many, the first surgery is the repair of the cleft palate. The majority of cleft palate patients suffer poor wound healing following surgery which leads to the development of an oro-nasal fistula (ONF) characterized by an opening between the oral and nasal cavities. One approach to repair an ONF is by using human donor tissue, which acts only as a physical barrier, and is not always effective. Thus, there is a need to identify improvements.

Ballestas et al. report improving hard palate wound healing using immune modulatory autotherapies. Acta Biomater, 2019, 91:209-219.

Toma et al. report oral wound healing models and emerging regenerative therapies. Transl Res, 2021, 236:17-34.

Darby et al. report Lactococcus lactis Subsp. cremoris is an efficacious beneficial bacterium that limits tissue injury in the intestine. iScience, 2019, 12:356-367

Amanso et al. report local delivery of FTY720 induces neutrophil activation through chemokine signaling in an oronasal fistula model. Regen Eng Transl Med, 2021, 7:160-174.

References reported herein are not an admission of prior art.

### SUMMARY

This disclosure relates to methods of improving tissue wound healing or growing tissues by using biodegradable graft compositions optionally comprising an exogenously added beneficial bacteria and optionally comprising exogenously added isolated macrophages or other cells. In certain embodiments, this disclosure relates to surgical methods of improving the healing of oral issues after a surgical intervention by implanting graft compositions reported herein on, in, or around the surgical site.

This disclosure relates to methods of improving tissue wound healing or growing tissues by using biodegradable graft compositions optionally comprising an exogenously added beneficial bacteria and optionally comprising exogenously added isolated macrophages, monocytes, or other cells. In certain embodiments, this disclosure relates to surgical methods of improving the healing of oral issues after a surgical intervention by implanting graft compositions reported herein on, in, or around the surgical site.

In certain embodiments, this disclosure relates to graft compositions comprising a graft matrix comprising an exogenously added beneficial microbe, secretions, or components therein or isolated or purified supernate derived therefrom, e.g., as a secretion derived from the beneficial microbe, and optionally macrophages, monocytes, and/or stem cells.

In certain embodiments, the beneficial microbe is selected from Lactococcus lactis subsp. cremoris (LLC) ATCC® 19257, Lancospiraceae sp. bacteria, Kineothrix alysoides (TSD-26®), Ruminococcaceae sp (TSD-27®), Lachnoclostridium phytofermentans (ATCC® 700394), Lactobacillus reuteri (ATCC® 6475), or combinations thereof.

In certain embodiments, this disclosure relates to macrophages or other cells as reported herein exposed to culture supernatant or ingredients from a pure culture of Lactococcus lactis subsp. cremoris (LLC) ATCC® 19257, and then delivery of those macrophages or cells into the ONF, wound healing site, mucosa, or graft, e.g., in PEG-4MAL hydrogel, to promote oral or other wound healing.

In certain embodiments, this disclosure relates to graft compositions comprising a graft matrix comprising an exogenously added beneficial microbe, secretions, or components therein or isolated supernate or components thereof and optionally macrophages, stem cells, and/or other cells reported herein.

In certain embodiments, this disclosure relates to a graft composition comprising a graft matrix comprising macrophages, stem cells and/or other cells. In certain embodiments, the macrophages or other cells are pre-exposed to 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof.

In certain embodiments, the graft matrix is biodegradable, e.g., biodegradable hydrogel. In certain embodiments, the hydrogel is a polyethylene glycol (PEG) based hydrogel. In certain embodiments, the polyethylene glycol-based hydrogel is a polyethylene glycol maleimide (PEG-MAL) hydrogel.

In certain embodiments, this disclosure relates to methods of closing an oro-nasal fistula comprising implanting an effective graft composition as disclosed herein into the oro-nasal fistula and/or onto the tissues or bone surrounding the oro-nasal fistula.

In certain embodiments, this disclosure relates to kits comprising a graft composition disclosed herein and optionally a beneficial bacterium, agents, macrophages and/or other cells used in methods reported herein.

### BRIEF DESCRIPTION OF THE SEVERAL VIEWS OF THE DRAWINGS

FIGS. 1A and 1B show data indicating delivery of a probiotic to the ONF wound site using hydrogel technology promotes oro-nasal fistula (ONF) wound healing. A hydrogel was tinted with a blue CDI tissue marking dye for enhanced observation. The hydrogel remains within the wound for 24 hours following application. In the experiments, the hydrogel containing probiotic can be reapplied every 24 hours. Representative images were obtained from mice inflicted with ONF, and then treated daily with polyethylene glycol maleimide (PEG-MAL) hydrogel only control, or a PEG-MAL hydrogel containing 1×10E6 CFU Lactococcus lactis subspecies cremoris (LLC). Note ONF is still visible in hydrogel only control mice after 7 days, but ONF is healed in mice treated with hydrogel containing 1×10E6 CFU LLC after 7 days. There was an expansion of Enterococcus faecalis in hydrogel only control mice, but markedly less E. faecalis detected in hydrogel containing LLC treated mice.

FIG. 1A shows measurement data of the Chao1 alpha diversity within oral microbiome samples of mice. Note significantly higher alpha diversity in mice treated with hydrogel containing LLC compared to control.

FIG. 1B shows the percentage of the total microbiome that is attributed to Enterococcus faecalis within the oral microbiome of samples. Note significantly lower amounts of E. faecalis in mice treated with hydrogel containing LLC compared to control.

FIG. 1C shows data indicating delivery of ThP1 cells (human monocytic cell line) exposed to LLC probiotic supernatant probiotic to the ONF wound site using hydrogel technology promotes ONF healing. Healing rates of ONF wounds in the palates of 12-week-old NOD/SCID mice following delivery of hydrogel only control, or hydrogel containing ThP1 macrophages exposed to filtered sterilized culture supernatant from a culture of LLC probiotic. Note ONF healed following therapy with ThP1 cells exposed to LLC. Quantitative analysis ONF healing at day 7 after wound infliction in 12-week-old NOD/SCID mice that received treatment of hydrogel alone, or hydrogel containing untreated ThP1 cells, or hydrogel containing ThP1 cells exposed to the filtered sterilized culture supernatant from a culture of the efficacious LLC probiotic, or the ineffectual LGG probiotic. Note HP-1 cells that had been exposed to LLC induce the nearly complete healing of the ONF in 9 out of 10 (90%) of mice. T test with p values stated. n=8, 6, 10 and 8 respectively.

FIG. 1D shows data indicating treatment of the human THP-1 macrophage line with supernatant from L. lactis cremoris induces cytokine expression. THP-1 macrophages were propagated in cell culture plates before treatment with filter sterilized supernatant from a culture of L. lactis cremoris (LLC) for 24 hours. Analysis show that LLC induces significant upregulation of G-CSF, IL-10 and IL-27 in THP-1 cells.

FIG. 2 illustrates contemplated ONF treatment methods of this disclosure. In one method, illustrated on the left, antibiotics are administered prior to surgery. Probiotics are included in the implant as a part of a hydrogel placed into the opening of the ONF to deliver the probiotics in the desired area of tissue growth. In a second method, illustrated on the right, macrophages are exposed to the supernate from the probiotics in vitro and transferred to the hydrogel which is implanted with immune cells into the ONF and surrounding tissues.

FIGS. 3A and 3B illustrate and show data from experiments to determine if FTY720P and macrophage hydrogel encapsulation promote oral wound healing in vivo.

FIG. 3A shows a schematic of PEG-4MAL hydrogel-macrophage preparation for in vivo analyses.

FIG. 3B shows data from representative endoscopic ONF images from an in vivo mouse model. Percent ONF wound closure was calculated on day 7 for mice treated with either 4% PEG-4MAL hydrogel blanks, hydrogels loaded with 264.7 macrophages, or macrophages pre-treated with FTY720P (10 μM).

### DETAILED DISCUSSION

Before the present disclosure is described in greater detail, it is to be understood that this disclosure is not limited to particular embodiments described, and as such may, of course, vary. An “embodiment” refers to an example, and it is not intended that the invention necessarily be limited by such example. It is also to be understood that the terminology used herein is for the purpose of describing particular embodiments only, and is not intended to be limiting, since the scope of the present disclosure will be limited only by the appended claims or as amended or added during prosecution.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

The term “graft composition” refers to materials that are substantially physiologically compatible when residing on a tissue, bone area, void, or exterior site. In certain embodiments, the graft composition may be a graft matrix such as a hydrogel, collagen sponge or a mixture of polymers and salts.

“Macrophages” refer to myeloid cells (white blood cells), derived from hematopoietic stem cells. Certain macrophages are reported to detect pathogen-associated molecular patterns (PAMPs), e.g., lipopolysaccharide (LPS) (bacterial cell wall component). Macrophages are often further classified into non-activated (M0), classically activated/pro-inflammatory (M1), or alternatively activated/anti-inflammatory (M2) subsets. Classically activated macrophages can be obtained in vitro by exposure to LPS and IFN-γ or TNF-α. M2 macrophage polarization can be induced by IL-4 or IL-13.

Solid tumors typically contain malignant cells and stromal host cells including macrophages, i.e., tumor associated macrophages (TAMs). Once differentiated, they can be polarized into M1 or M2 macrophages that express markers similarly to polarized macrophages obtained from freshly isolated monocytes. ATCC® RAW 264.7 monocyte/macrophage-like cells originate from Abelson leukemia virus transformed cell line derived from BALB/c mice. Macrophages can also be obtained from the human monocytic cell line THP1 (ATCC® TIB-202), a human leukemia monocytic cell line, by exposure to PMA. PMA-stimulated differentiation is generally preferred for generation of cells with similarities to human peripheral blood mononuclear cell (PBMC) monocyte-derived macrophages.

The terms “exogenously added” in reference to a component of a mixture refers to a component that does not naturally originate specifically from one of the other components in a mixture. This should not be taken to mean that exogenously added component may not be of natural origin. For example, tissue may produce a polypeptide and a dehydrated tissue may contain the polypeptide. However, dehydrated tissue comprising an exogenously added polypeptide is not referring to the polypeptide that was produced by the tissue or a polypeptide that exists in the tissue component after dehydration. An exogenously added polypeptide may be synthesized, isolated, or purified external from a different sample of the same tissue which is then added to the tissue or dehydrated tissue component.

As used herein, the term “biodegradable” refers to a material that when transplanted into an area of a subject, e.g., human, will be degraded by biological mechanism such that the material will not persist in the area for over a long period of time, e.g., material will be removed by the body after a couple weeks or months. In certain embodiments, the disclose contemplates that the biodegradable material will not be found at the transplanted location after six months, a year, or two years.

As used herein, a “subject” refers to any animal, preferably a human patient, rodent, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g., patient) is cured and the disease is eradicated. Rather, embodiments of the present disclosure also contemplate treatment that merely reduces symptoms, and/or delays disease progression.

### Grafting Methods and Timing of Anti-Microbial Therapy

The “oralome” refers to inter-species and host-microbial interactions that take place in the oral cavity which can be further defined as “eubiotic” healthy or “dysbiotic” disease states. Wound healing in the mouth is sensitive to local environmental factors, including the oralome, where certain microbial community structures or probiotic bacteria can impede or enhance the wound healing process.

Although it is not intended that certain embodiments, of this disclosure be limited by any particular mechanisms, it is believed that eubiotic oral microbiome exerts positive modulatory influences on oral wound healing through immune events that can be harnessed; thus, a dysbiotic oral microbiome state prior to surgery negatively impacts oral wound healing, and the use of a probiotic therapy that guides tissue regeneration via the modulation of macrophages promotes oral wound healing after surgery.

In certain embodiments, this disclosure relates to method of performing a surgical procedure that includes making incisions, e.g., in the oral cavity, and administering a multiple antibiotic therapy (two, three, four, or more antibiotics) more than two, three or four days before the surgery (incision), and not administering any antibiotic therapy for at least after 24 hour, 48 hours, or 72 hours, before commencement of the surgery. In certain embodiments, this disclosure relates to method of performing a surgy and not administering an antibiotic therapy during the surgery, and not administering an antibiotic therapy for at least after 48 hours, 72 hours, or 98 hours after commencement of the surgery.

In certain embodiments, the methods further comprise administering to the patient or implanting in the patient a composition or biodegradable substance comprising a beneficial microbe, secretions, or components therein. In certain embodiments, administering to the patient or implanting in the patient a composition or biodegradable substance comprising a beneficial microbe, secretions, or components therein is after commencement of the surgery, 24 hours before surgery, and or 24 hour, 48 hours, or 72 hours after the surgery. In certain embodiments, the beneficial microbe, secretions, or components therein is administered on the day of surgery or after the surgery once daily or twice daily for two or more days, three or more days, four or more days, five or more days.

In certain embodiments, the beneficial microbe is selected Lactococcus lactis subsp. cremoris (LLC) ATCC® 19257, Lancospiraceae sp. bacteria, Kineothrix alysoides (TSD-26®), Ruminococcaceae sp (TSD-27®), Lachnoclostridium phytofermentans (ATCC® 700394), Lactobacillus reuteri (ATCC® 6475), or combinations thereof.

In certain embodiments, the surgery is an oral cavity surgery. In certain embodiments, the surgery is closing a cleft palate or cleft lip to prevent the formation of or reduce the size of an oro-nasal fistula. In certain embodiments, this disclosure contemplates using graft compositions disclosed herein during the surgery.

It is also contemplated that dysbiotic oral microbiome impairs oral wound healing due to prolonged inflammation which can be evidenced by correlative ONF healing rates with changes in the bacterial, fungal, and viral microbiome. It is contemplated that probiotic induced enhanced ONF healing occurs due to the secretion of microbial factors that promote macrophage activity within injured tissue. Thus, in certain embodiments, this disclosure relates to therapeutic and surgical methods wherein a probiotic is introduced to the ONF site, e.g., touching the surrounding tissues or bones within a PEG-MAL hydrogel comprising agents disclosed herein.

This disclosure relates to methods of improving oral tissue wound healing or growing tissues by using biodegradable graft compositions optionally comprising an exogenously added beneficial microbe, secretions, or components therein, and optionally comprising exogenously added isolated macrophages or stem cells. In certain embodiments, this disclosure relates to surgical methods of improving the healing of oral issues after a surgical intervention (incising oral tissues) by implanting graft compositions reported herein on, in, or around the surgical site.

In certain embodiments, this disclosure relates to hydrogel delivery of probiotics and activated macrophages or other cells to enhance closure of an ONF and improve wound healing. It is contemplated that probiotics promote optimal ONF healing by preferentially activating macrophages or other cells and that hydrogel delivery of a probiotic improves ONF healing through immunomodulation.

Cleft palate repair involves separation of the oral and nasal cavities, that is surgically achieved by elevating and rotating the palate tissues to the midline. Successful natural healing of these rotated mucosal flaps depends upon minimizing wound tension, gaps, and inflammation. Using local or free tissue flaps are often the last resort strategy, including the attachment of tongue tissue to the ONF or transfer of a free tissue flap from the radius of the thigh.

One object of this disclosure is to control inflammation in order to optimize mucosal wound healing, e.g., oral wound healing and other areas of mucosa like intestines and reduce the need for surgical ONF revision surgeries and ensure healing in patients who need surgical revision ONF surgeries. Wound healing of the oral cavity is typically faster than other cutaneous wound healing. There is less scar tissue formation, and oral wound healing happens even though healing in the oral cavity occurs in an environment that undergoes constant trauma and within an environment suitable for microbial growth.

Wound healing following cleft palate repair involves separation of the oral and nasal cavities that is surgically achieved by elevating and rotating the palate tissues to the midline. Many factors that are associated with the occurrence of an ONF play a role in healing of these rotated mucosal flaps, including wound tension, width of the cleft palate, and prolonged, persistent inflammation. To mitigate the occurrence of ONF formation, surgeons may implant a physical barrier (donated human dermis) to improve wound healing or add an additional layer of the closure to reduce ONF formation. Local or free tissue flaps, including the attachment of tissue to the ONF or transfer of a free tissue flap from the radius, tongue, or the thigh. Controlling inflammation to optimize oral wound healing is desirable to reduce the need for surgical ONF revision surgeries and ensure healing in patients who need surgical revision ONF surgeries.

Tissue repair occurs during the inflammatory stage which is contingent on the mobilization of circulating monocytes into the wound bed. Based on local factors, these monocytes undergo differentiation into inflammatory monocytes (IM) and anti-inflammatory monocytes (AM). Monocytes, which then become macrophages, can be categorized by many markers, including the surface marker Ly6C. In mice, the IMs (identified as Ly6C^{hi}) secrete large amounts of TNFα and IL-1 to stimulate phagocytosis, whereas the AMs (identified as Ly6C^{lo}) secrete large amounts of IL-4 and IL-10 that stimulate regeneration.

In certain embodiments, this disclosure relates to a surgical method of treating ONF comprising: administering an antibiotic to a patient prior to surgery: cutting tissue of the patient to provide tissue for inducing the natural healing of inflamed tissue; implanting a hydrogel into the opening of the ONF comprising probiotics and optionally adding a composition comprising probiotics to the opening of or the area around the ONF; and allowing the healing of the ONF to provide closure of the ONF, i.e., providing a tissue or scar barrier between the mouth and the nasal cavity. In certain embodiments, methods comprising adding macrophages or other cells to a hydrogel that are exposed to probiotic in vitro and implanting the hydrogel into or in contact with the ONF or surrounding tissue.

In certain embodiments, this disclosure relates to a surgical method of treating ONF comprising: administering an antibiotic to a patient prior to surgery: cutting tissue of the patient to provide tissue for inducing the natural healing of inflamed tissue; implanting a hydrogel comprising adding macrophages or other cells that are exposed to a probiotic or secretions or components in the secretions into the opening of the ONF comprising probiotics and optionally adding a composition comprising probiotics or secretions or components in the secretions to the opening of or the area around the ONF; and allowing the healing of the ONF to provide closure of the ONF, i.e., providing a tissue or scar barrier between the mouth and the nasal cavity. In certain embodiments, methods comprising adding macrophages or other cells to a hydrogel that are exposed to probiotic, secretions, or components therein in vitro and implanting the hydrogel into or in contact with the ONF or surrounding tissue.

In certain embodiments, this disclosure relates to isolated macrophages or other cells reported herein exposed to culture supernatant of Lactococcus lactis subsp. cremoris (LLC) ATCC® 19257 or exposed to components therein and delivering the macrophages within PEG-4MAL hydrogels into the oral-nasal-facial (ONF) region or other wound.

In certain embodiments, this disclosure relates to methods of growing tissues and/or surrounding bone by using/implanting graft compositions disclosed herein in contact with or near tissues with an oral wound, e.g., following trauma and/or surgery (cancer removal of a tumor) in the oral cavity on tissues that coat the mandible, maxilla, or palatine bones.

In certain embodiments, this disclosure relates to methods of closing an oro-nasal fistula (ONF) comprising implanting a graft composition as reported herein into the oro-nasal fistula and/or surrounding tissues. In certain embodiments, the graft composition comprises an exogenously added beneficial microbe or exogenous secretions of the beneficial microbe or exogenous components therein. In certain embodiments, the beneficial microbe is selected Lactococcus lactis subsp. cremoris (LLC) ATCC® 19257, Lancospiraceae sp. bacteria, Kineothrix alysoides (TSD-26®), Ruminococcaceae sp (TSD-27®), Lachnoclostridium phytofermentans (ATCC® 700394), Lactobacillus reuteri (ATCC® 6475), or combinations thereof.

In certain embodiments, the graft composition comprises exogenously added 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof.

In certain embodiments, the graft composition comprises exogenously added macrophages and/or stem cells pre-exposed to 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof or exogenously added macrophages and 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof in the graft composition.

In certain embodiments, this disclosure relates to methods of forming tissue and/or bone comprising implanting an effective graft composition as reported herein in a subject under conditions such that tissue or bone forms in the graft composition. In certain embodiments, the subject has a void in the tissue or bony structure wherein the graft composition is implanted in the void wherein tissue and/or bone grows in or around the void. In certain embodiments, the void is in a bone of the maxilla, face, palatine bones, or cranium. In certain embodiments, the void is a result of an orofacial cleft, cleft palate, cleft lip, or surgical removal of bone. In certain embodiments, the void is due to surgical removal of tissue and/or bone, e.g., due to the presence and removal of a tumor or bone cancer.

In certain embodiments, this disclosure relates to surgical methods of closing a surgical wound, oro-nasal fistula (ONF), traumatic wounds, and/or and cancer surgical wounds by implanting graft compositions reported herein over the ONF and around the surrounding tissues.

In certain embodiments, this disclosure relates to surgical methods of incising an oral mucosa to correct cellulitis or periodontal abscess opening, gingival tissue excision or repositioning or exposure of alveolar bone for tooth extraction or surgical removal of soft tissue between the tooth root and bone (infrabony technique) and implanting a graft composition reported herein to improve wound healing.

In certain embodiments, for any of the methods disclosed herein administering/implanting the macrophages or other cells is in combination with administering/implanting exogenously added 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof to the subject. In certain embodiments, the methods further comprising administering transforming growth factor-beta (TGF-β) type III receptor and/or a bone morphogenetic protein, e.g., BMP-2 or other agent.

In certain embodiments, the disclosure relates to a method of forming tissue or bone comprising implanting a graft composition optionally comprising an exogenously added beneficial bacteria and/or optionally comprising exogenously added isolated macrophages, stem cells, induced pluripotent stem cells or other cells as reported herein in a subject. In certain embodiments, the disclosure relates to methods of forming tissue or bone comprising implanting a graft composition comprising exogenously added isolated macrophages, stem cells, or induced pluripotent stem cells, a bone morphogenetic protein and/or TβRIII in a subject.

In certain embodiments, the graft may be the result of a void created by surgical removal or created as a result of an attempt to correct a physical abnormality, such as but not limited to, a cleft palate, cleft lip in order to prevent the formation of or reduce the size of an oro-nasal fistula (ONF). In certain embodiments, this disclosure relates to surgical methods of closing a cleft palate and/or cleft lip in order to prevent the formation of or reduce the size of an oro-nasal fistula (ONF). In certain embodiments, this disclosure relates to methods of surgically correcting a cleft palate or lip comprising implanting an effective graft composition as disclosed herein into the tissues of the oral cavity and/or onto the tissues or bone surrounding the oral cavity or surgical site or other mucosal tissues.

### Graft Compositions

In certain embodiments, this disclosure relates to graft compositions comprising a graft matrix comprising an exogenously added beneficial microbe, secretions, or components therein and optionally exogenously added and/or encapsulated macrophages, induced pluripotent stem cells, adult stem cells, stem cells, hematopoietic cells, and/or other cells. In certain embodiments, this disclosure relates to a graft matrix comprising macrophages, stem cells, induced pluripotent stem cells, adult stem cells, hematopoietic cells, and/or other cells and optionally a beneficial microbe or microbe secretions or components of the secretions.

In certain embodiments, the beneficial microbe is selected from Lactococcus lactis subsp. cremoris (LLC) ATCC® 19257, Lancospiraceae sp. bacteria, Kineothrix alysoides (TSD-26®), Ruminococcaceae sp (TSD-27®), Lachnoclostridium phytofermentans (ATCC® 700394), Lactobacillus reuteri (ATCC® 6475), or combinations thereof.

In certain embodiments, the macrophages, stem cells, induced pluripotent stem cells, adult stem cells, hematopoietic cells, and/or other cells are autologous or non-autologous. In certain embodiments, the macrophages or other cells are replicated ex vivo.

In certain embodiments, the cells are human peripheral blood mononuclear cells (PBMC), monocyte-derived macrophages, or stem cells. In certain embodiments, the macrophages are CD45+ enriched M2 macrophages. In certain embodiments, the macrophages are CD14+ enriched M2 macrophages. In certain embodiments, the macrophages are CD45+ and CD14+ enriched M2 macrophages.

In certain embodiments, the macrophages are hematopoietic progenitors that express CD14+ CD45+ monocytes derived from induced pluripotent stem cell (iPSC). See Park et al. Differentiation of monocytes and polarized M1/M2 macrophages from human induced pluripotent stem cells. STAR Protocols 5, 102827, 2023.

In certain embodiments, the macrophages are ixmyelocel-T cells, i.e., an expanded, autologous cells cultured from bone-marrow mononuclear cells. In certain embodiments, the cells are a mixed population of bone marrow mononuclear cells containing CD90+ enriched mesenchymal stem cells (MSCs) and CD45+ and CD14+ enriched M2 macrophages. In certain embodiments, the cells are a mixed population of cells of hematopoietic, mesenchymal, and endothelial lineage, wherein the composition contains about 5-75% viable CD90+ cells with the remaining cells in said composition being CD45+. In certain embodiments, CD45+ cells are CD14+. See also U.S. Pat. No. 9,415,071, hereby incorporated by reference.

In certain embodiments, the macrophages, stem cells, and/or other cells are optionally pre-exposed ex vivo to 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof. In certain embodiments, the macrophages are exposed to 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof as a component of the graft matrix, e.g., graft composition comprises 2-amino-2-[2-(4-octyl-phenyl)-ethyl]-propane-1,3-diol (Fingolimod/FTY720), 2-amino-2-(hydroxymethyl)-4-(4-octyl phenyl)butyl dihydrogen phosphate (Fingolimod phosphate/FTY720P), prodrug, or salt thereof. In certain embodiments, the graft composition further comprises exogenously added transforming growth factor-beta (TGF-β) type III receptor and or a bone morphogenetic protein, e.g., BMP-2 or other.

In certain embodiments, the macrophages, stem cells, and/or other cells are optionally pre-exposed ex vivo to the beneficial microbe, secretions, or components therein.

In certain embodiments, the hydrogel is a polyethylene glycol (PEG) based hydrogel. In certain embodiments, the polyethylene glycol-based hydrogel is polyethylene glycol maleimide (PEG-MAL) hydrogel. In certain embodiments, the graft matrix is a hydrogel, collagen, or demineralized bone.

In some embodiments, the disclosure relates to graft compositions comprising an exogenously added beneficial microbe, secretions, or components therein and optionally encapsulated cells and optionally other growth factor(s). In certain embodiments, the graft may be a porous or non-porous material that comprises or is coated with compositions disclosed herein.

In certain embodiments, a hydrogel may contain polyethylene glycol or polyethylene glycol macromers or dendrimers terminally substituted with reactive coupling groups, e.g., PEG-diacrylate, 4-arm PEG-maleimide (PEG-4MAL) macromere, 4-arm PEG-acrylate (PEG-4A), 4-arm PEG-vinyl sulfone (PEG-4VS). The macromers or dendrimers may be further functionalized with crosslinking agents or polypeptides with desirable functional attributes, e.g., the graft or hydrogel may be designed to contain an adhesive RGD sequence or a protease-cleavable peptide sequence to facilitate the biodegradable nature of an implanted composition.

In certain embodiments, the disclosure relates to graft compositions comprising agents, e.g. 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof, BMP-2, TβRIII, which are covalently linked or are not covalently linked to graft compositions or scaffolds. In some embodiments, these compositions may be combined with growth factor(s).

In some embodiments, the disclosure relates to a graft composition comprising an exogenously added beneficial microbe, secretions, or components therein, microphages, cells, agents, e.g. 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof, BMP-2, TβRIII, and/or polysaccharides such as hyaluronate, alginate, cellulose, or cellulose derivatives such as, but not limited to, hydroxypropyl cellulose, methyl cellulose, ethyl cellulose, and carboxymethyl cellulose. Typically, cellulose derivatives are used in graft compositions that produce a paste or putty.

In some embodiments, the disclosure relates to graft compositions comprising exogenously added beneficial microbe, secretions, or components therein, microphages, cells, agents, e.g., 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof, BMP-2, TβRIII, and/or polysaccharides and a graft matrix. In certain embodiments, the matrix is a polymer designed to hold compatible salts, such as calcium phosphates, e.g., a bovine Type I collagen embedded with biphasic calcium phosphate granules. Optionally, matrix compositions may also include one or more agents such as alkaline phosphatase, osteocalcin, bone sialoprotein (BSP) and osteocalcin, phosphoprotein (SPP)-1, type I collagen, fibronectin, osteonectin, thrombospondin, matrix-gla-protein (MGP), SPARC, and osteopontin.

In certain embodiments, the graft matrix can be made up of a hydrogel polymer. Typically, a hydrogel is made-up of polymers and copolymers substituted with an abundance of hydrophilic groups, such as polyethylene glycol and/or terminal hydroxyl or carboxyl groups. In certain embodiments, the graft composition is biodegradable. In certain embodiments, the graft matrix is a hydrogel, demineralized bone, or collagen. In certain embodiments, the hydrogel is a polyethylene glycol (PEG) based hydrogel. In certain embodiments, the polyethylene glycol-based hydrogel is polyethylene glycol maleimide (PEG-MAL) hydrogel. In certain embodiments, the matrix comprises homopolymers and copolymers consisting of glycolide and lactide. Such a composition may also be made with crosslinkers comprising an ester, anhydride, orthoester, amide, and/or peptide bonds. In some embodiments, crosslinkers contain the following polymers: polyethylene glycol (PEG), polylactic acid, polyglycolide or combinations thereof.

In certain embodiments, graft comprises recombinant human platelet-derived growth factor (becaplermin).

In certain embodiments, the graft composition may contain one or more antibiotics and/or anti-inflammatory agents. Suitable antibiotics include, without limitation, such as 2,4-diaminopyrimidines, including baquiloprim, brodimoprim, iclaprim, ormetoprim, pyrimethamine, tetroxoprim, trimethoprim; aminocoumarins, including novobiocin; aminocyclitols, including spectinomycin; aminoglycosides, including amikacin, apramycin, arbekacin, bekanamycin, butirosin, dibekacin, dihydrostreptomycin, etimicin, fortimicins, astromicin, framycetin, gentamicin, hygromycin B, isepamicin, kanamycin, micronomicin, neomycin, netilmicin, paromomycin, plazomicin, ribostamycin, sisomicin, streptomycin, tobramycin; aminomethylcyclines, including omadacycline; amphenicols, including azidamfenicol, chloramphenicol, florfenicol, thiamphenicol; ansamycins, including rifabutin, rifampicin, rifapentine, rifaximin; antitubercular agents, including cycloserine, delamanid, ethambutol, ethionamide, morinamide, p-aminosalicylic acid (PAS), protionamide, pyrazinamide, terizidone, thioacetazone; bacteriocins, including nisin; b-lactam carbapenems, including loracarbef biapenem, doripenem, ertapenem, faropenem, imipenem, meropenem, panipenem, razupenem, sulopenem, tebipenem, tomopenem; b-lactam cephalosporins, including cefacetrile, cefaclor, cefadroxil, cefalexin, cefalonium, cefaloridine, cefamandole, cefapirin, cefatrizine, cefazaflur, cefazedone, cefazolin, cefcapene, cefdinir, cefditoren, cefepime, cefetamet, cefixime, cefmenoxime, cefodizime, cefonicid, cefoperazone, ceforanide, cefotaxime, cefotiam, cefovecin, cefozopran, cefpimizole, cefpiramide, cefpirome, cefpodoxime, cefprozil, cefquinome, cefradine, cefroxadine, cefsulodin, ceftaroline, ceftazidime, cefteram, ceftezole, ceftibuten, ceftiofur, ceftizoxime, ceftobiprole, ceftolozane, ceftriaxone, cefuroxime, cefuzonam; b-lactam cephamycin, including cefbuperazone, cefmetazole, cefotetan, cefoxitin; b-lactam monobactams, including aztreonam, carumonam, tigemonam; b-lactam oxacephems, including flomoxef, latamoxef, moxalactam; b-lactam penicillins, including amdinocillin, amoxicillin, ampicillin, apalcillin, azidocillin, azlocillin, bacampicillin, carbenicillin, ciclacillin, clemizole penicillin, cloxacillin, cyclacillin, dicloxacillin, epicillin, fenbenicillin, floxacillin, hetacillin, mecillinam, metampicillin, methicillin sodium, mezlocillin, nafcillin, oxacillin, penamecillin, penethamate hydroiodide, penicillin G, penicillin G benzathine, penicillin G procaine, penicillin N, penicillin O, penicillin V, phenethicillin potassium, piperacillin, pivampicillin, pivmecillinam, propicillin, quinacillin, sultamicillin, talampicillin, temocillin, ticarcillin; b-lactam in combination with b-lactamase inhibitors, including avibactam, clavulanic acid, relebactam, sulbactam, tazobactam, vaborbactam, zidebactam; bicyclomycins, including bicozamycin; bis-benzimidazoles including ridinilazole; cyclic esters, including fosfomycin; fluoroquinolones, including avarofloxacin, balofloxacin, besifloxacin, cinoxacin, ciprofloxacin, clinafloxacin, danofloxacin, delafloxacin, difloxacin, enoxacin, enrofloxacin, finafloxacin, fleroxacin, flumequine, garenoxacin, gatifloxacin, gemifloxacin, grepafloxacin, levofloxacin, lomefloxacin, marbofloxacin, miloxacin, moxifloxacin, nadifloxacin, norfloxacin, ofloxacin, orbifloxacin, pazufloxacin, pefloxacin, pradofloxacin, prulifloxacin, rosoxacin, rufloxacin, sarafloxacin, sitafloxacin, sparfloxacin, temafloxacin, tosufloxacin, trovafloxacin, zabofloxacin; fusidane-type, including helvolic acid, fusidic acid and cephalosporin P1; glycolipodepsipeptides, including ramoplanin; glycopeptides, including avoparcin, dalbavancin, norvancomycin, oritavancin, teicoplanin, telavancin, vancomycin; glycylcyclines, including tigecycline; hybrids, oxazolidinone-quinolone, TD-1792 (glycopeptide-cephalosporin); lincosamides, including clindamycin, lincomycin, pirlimycin; lipopeptides, including daptomycin, surotomycin; macrolides, including azithromycin, cethromycin, clarithromycin, dirithromycin, erythromycin, fidaxomicin, flurithromycin, gamithromycin, josamycin, kitasamycin, leucomycin, miocamycin, oleandomycin, primycin, rokitamycin, rosaramicin, roxithromycin, solithromycin, spiramycin, telithromycin, tildipirosin, tilmicosin, troleandomycin, tulathromycin, tylosin, tylvalosin; nitrofurans, including furaltadone, furazidin, furazolidone, nifuroxazide, nifurtoinol, nifurzide, nitrofural, nitrofurantoin, nitrofurazone; nitroimidazoles, including dimetridazole, metronidazole, ornidazole, secnidazole, tinidazole; oligosaccharides, including avilamycin, everninomicin; polymyxins, including polymyxin E (colistin), polymyxin B; polypeptides, including amphomycin, bacitracin, capreomycin, enduracidin, enramycin, gramicidin(s), ristocetin, tuberactinomycin, tyrocidine, tyrothricin, viomycin; pseudomonic acids including mupirocin; quinoxalines including carbadox, olaquindox; sulphonamides including acetyl sulfamethoxypyrazine, chloramine-B, chloramine-T, dichloramine T, mafenide, sulfacetamide, sulfadiazine, sulfadimidine, sulfamethazine, sulfamethizole, sulfapyridine, sulfathiazole, sulfisomidine, sulfisoxazole; sulfones, including dapsone sodium, sulfanilic acid; tetracycline including chlortetracycline, clomocycline, demeclocycline, doxycycline, lymecycline, meclocycline, methacycline, minocycline, oxytetracycline, rolitetracycline, tetracycline; triazaacenaphthylene including gepotidacin, or any combination thereof.

Suitable anti-inflammatory compounds include both steroidal and non-steroidal structures. Suitable non-limiting examples of steroidal anti-inflammatory compounds are corticosteroids such as hydrocortisone, cortisol, triamcinolone, alpha-methyl dexamethasone, dexamethasone-phosphate, beclomethasone dipropionates, clobetasol valerate, desonide, desoximetasone, desoxycorticosterone acetate, dexamethasone, diflorasone diacetate, diflucortolone valerate, flurandrenolone, fluclorolone acetonide, fludrocortisone, flumethasone pivalate, fluocinolone acetonide, fluocinonide, fluocortin butyl ester, fluocortolone, fluprednidene acetate, flurandrenolone, halcinonide, hydrocortisone acetate, hydrocortisone butyrate, methylprednisolone, triamcinolone acetonide, cortisone, cortodoxone, fludrocortisone, fludrocortisone, fluocinolone, medrysone, betamethasone and the balance of its esters, chloroprednisone, dichlorisone, flunisolide, fluperolone, fluprednisolone, hydrocortisone valerate, hydrocortamate, prednisone, paramethasone, prednisolone, prednisone, beclomethasone dipropionate, and triamcinolone. Mixtures of the above steroidal anti-inflammatory compounds may also be used.

Non-limiting examples of non-steroidal anti-inflammatory compounds include nabumetone, celecoxib, etodolac, nimesulide, gold, oxicams, such as piroxicam, isoxicam, meloxicam, tenoxicam, sudoxicam, the salicylates, such as aspirin, disalcid, benorylate, trilisate, safapryn, diflunisal, and fendosal; the acetic acid derivatives, such as diclofenac, fenclofenac, indomethacin, sulindac, tolmetin, isoxepac, furofenac, tiopinac, zidometacin, fentiazac, zomepirac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

In certain embodiments, a graft matrix may be loaded with cells or agents during manufacturing. In certain embodiments, the graft matrix is loaded with cells or agents just prior to implantation.

### Kits and Pharmaceutical Packaging

In certain embodiments, this disclosure contemplates a kit comprising a hydrogel disclosed herein and optionally a container comprising beneficial microbe, secretions, or components therein and optionally multiple antibiotic agents and optionally macrophages and/or other cells reported herein. In certain embodiments, the beneficial microbe is selected from Lactococcus lactis subsp. cremoris (LLC) ATCC® 19257, Lancospiraceae sp. bacteria, Kineothrix alysoides (TSD-26®), Ruminococcaceae sp (TSD-27®), Lachnoclostridium phytofermentans (ATCC® 700394), Lactobacillus reuteri (ATCC® 6475), or combinations thereof. In certain embodiments, the kit comprises multiple individual and different antibiotic agents in the same or separate containers.

Further components of the kit may be instructions for use, administration means, such as a sterilized syringe, catheter, pipette, tweezer, brush, etc. or other components necessary for use in medical (surgical) practice, such as substitute needle, catheter, pipette, pipette tip, extra container, vial, bandage, tape, or further wound cover means.

In certain embodiments, the kit comprises graft matrix, a hydrated, dehydrated, or partially dehydrated hydrogel graft matrix reported herein or and stable hemostatic graft matrix and optionally a syringe containing the diluent (or provided to take up the diluent from another diluent container) and apply to the hydrogel or other graft matrix.

### EXAMPLES

### Antibiotic Treatment Impacts the Oral Microbiome During Oro-Nasal Fistula (ONF) Wound Healing

E. faecalis is associated with oral diseases, such as caries, endodontic infections, and periodontitis. However, the implications of these changes to the oral microbiome composition, both loss of taxa diversity or alpha diversity, and the bloom of other taxa such as E. faecalis, to tissue healing processes remains a mystery. Moreover, whether interactions of specific oral microbes with host cells activate pro-restitutive cellular signaling pathways in the oral wound bed are also unknown. To this end, mice were treated with a cocktail of 1 mg/mL ampicillin, 0.5 mg/mL vancomycin, 1 mg/mL neomycin, 1 mg/mL metronidazole included in their drinking water, which are absorbable and non-absorbable antibiotics commonly used in experimental science to deplete the murine microbiome of normal viability, for 1 week. Greater than 99.9% lowering in total bacterial burden was detected. An expected reduction in the microbiome alpha diversity was also observed. Antibiotic treatment rendered E. faecalis undetectable in the oral microbiome at day 0 (before ONF), and throughout the 7 days over which the rate of healing is followed. However, the antibiotic-mediated elimination of the bacterial load in the oral cavity did not impact ONF healing, with ONF still visible in the palate of mice at day 7 after wound infliction. These observations suggest that antibiotics impact oral microbiome composition, but do not promote wound healing. The data collectively suggest that a dysbiotic microbiome, or loss of microbiome diversity may both impair wound healing and may contribute to the formation of an ONF.

Lactococcus lactis Subsp. Cremoris (LLC) in the Wound Bed of the Freshly Inflicted Oro-Nasal (ONF) Fistula Via a PEG-MAL Hydrogel

Experiments reported herein indicate adding Lactococcus lactis subsp. cremoris (LLC) to the wound bed of the freshly inflicted ONF via a PEG-MAL hydrogel vehicle resulted in rapid healing of the ONF. After implantation of the hydrogel into an ONF wound bed, mice were treated with antibiotics, dysbiotic bacteria, and hydrogel-delivered pro-tissue restitutive probiotics for disrupting and restoring the oral microbiome during ONF healing. It is contemplated that macrophage-based therapy enhances ONF healing.

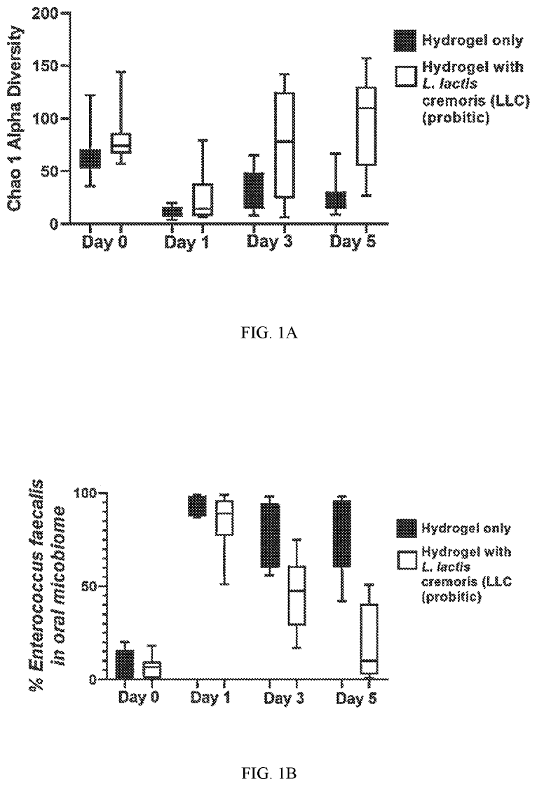

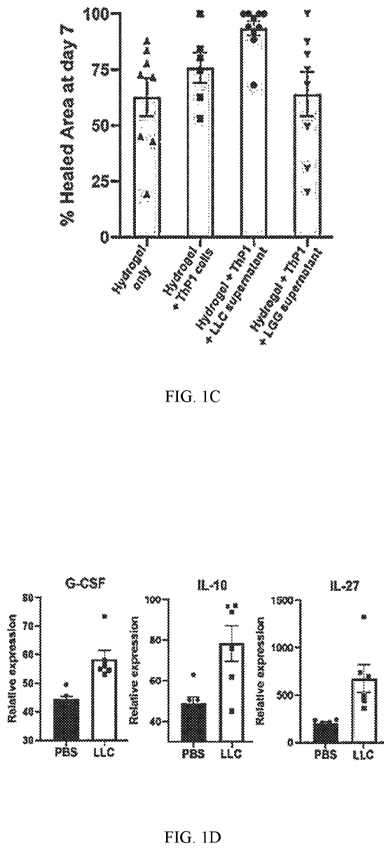

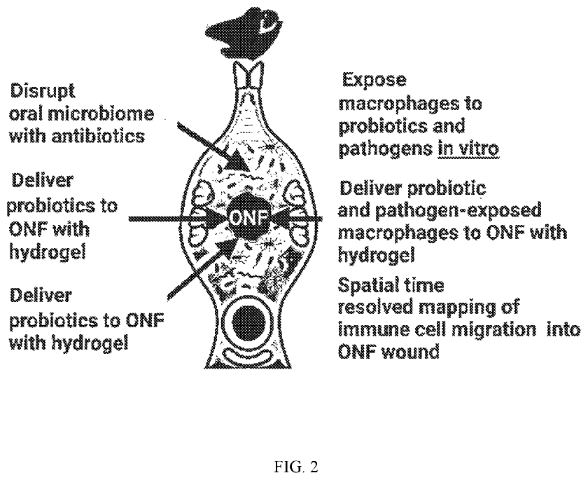

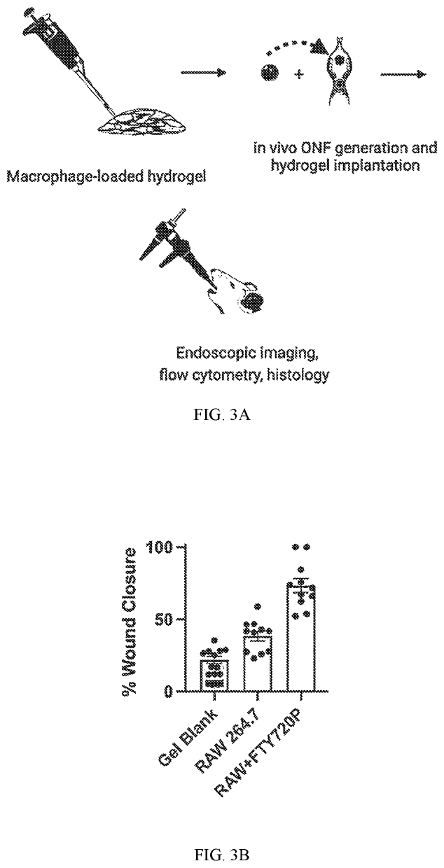

Experiments were performed that indicate antibiotics administered during oral surgery lowers the microbial burden in the oral cavity but does not enhance wound healing of an ONF. Strikingly, delivery of a pro-regenerative beneficial microbe, LLC into the ONF resulted in complete healing of the ONF and restored the microbial diversity (FIGS. 1A and 1B). This indicates that restoring an eubiotic oral microbiome exerts positive modulatory influences on oral wound healing, plausibly through bacterial-bolstered immune and epithelial cell regenerative cues.

A step of tissue repair occurs during the inflammatory stage, and this is contemplated to be dependent on the mobilization of circulating monocytes into the wound bed. Based on local factors, these monocytes undergo differentiation into inflammatory (IM) and anti-inflammatory (AM) monocytes. It is contemplated that IM and AM are sensitive to the quantity and quality of the oral microbiome composition. Relevant to ONF healing is a notion that a dysbiotic oral microbiome detected following a ONF infliction is one that is enriched in E. faecalis, S. lentus, and S. xylosus which may provoke an aggravated immune response that impedes tissue healing.

On the other hand, elimination of a commensal background microbiome using a broad-spectrum antibiotic cocktail, although eliminating E. faecalis, S. lentus, and S. xylosus, did not improve ONF healing. Indeed, data suggest that a near complete absence of bacteria (99.9% less bacterial load than untreated) and blunting of an immune cell response does not result in a pro-restitutive wound healing events in the oral tissue. However, delivery of a specific strain of probiotic bacteria, i.e., LLC, directly into the ONF via a hydrogel promotes ONF healing.

Macrophages Treated with FTY720-Phosphate in PEG-4MAL Hydrogels Promote Oral Wound Healing

Polyethylene glycol (PEG)-based synthetic hydrogels were prepared by first reacting, maleimide end-functionalized (four-arm) PEG macromer (PEG-4MAL) (4ARM-PEG-MAL-20K) with a thiol-containing adhesive peptide GRGDSPC (SEQ ID NO: 1) in PBS with 20 mM HEPES (pH 7.4) for 1 hour. RGD-functionalized PEG-4MAL macromers were cross-linked in the presence of either murine RAW 264.7 or human M0-differentiated THP1 cells by adding a peptide GPQ and W cross-linker [amino acid sequence, GCRDGPQGIWGQDRCG (SEQ ID NO: 2)]. The final PEG-4MAL hydrogel cell-encapsulation consisted of 4.0% (wt/vol) polymer and 1.0 mM RGD and contained 50,000 cells per μL.

To differentiate THP1 cells into M0 macrophages, cells were treated with PMA (Phorbol 12-myristate 13-acetate) for 48 hours and subsequentially harvested using trypsin. Prior to casting cell-hydrogel encapsulations, macrophages were exposed to FTY720P. RAW 264.7 (monocyte/macrophage-like cells, originating from Abelson leukemia virus transformed cell line derived from BALB/c mice) or M0-differentiated THP1 cells were pre-treated with select concentrations of FTY720P for 1 hour at 37° C.

Experiments were performed to determine whether pre-treatment of macrophages with phosphorylated FTY720 (FTY720P) in a PEG-4MAL hydrogel vehicle encapsulation may promote oral wound healing and tissue regeneration. FTY720P-treated macrophages in hydrogel promoted a pro-regenerative environment and secreted chemokines and cytokines that induce M2 polarization, migration of macrophages and fibroblasts, angiogenesis, and epithelization. Moreover, drug-treated cells induced chemotaxis of macrophages, fibroblasts, and scratch wound closure in oral epithelial cells in vitro. In a murine oronasal fistula model of oral wound healing, local application of FTY720P-treated macrophages in hydrogel resulted significantly increased wound closure relative to controls. These results indicate clinical implications for cleft palate and oral wound healing.

Macrophages Exposed to Culture Supernatant from a Pure Culture of Lactococcus lactis Subsp. Cremoris (LLC) ATCC® 19257, and then Delivery of the LLC Exposed Macrophage into the ONF in PEG-4MAL Hydrogels Promote Oral Wound Healing.

Experiments were performed to determine whether delivery of cultured macrophages via hydrogel to the wound bed could impact ONF healing. Furthermore, some macrophages were incubated for 24 hours in the presence of diluted filtered medium from cultures of probiotic bacteria. Specifically, for clinical relevance, suspensions of human THP-1 macrophages were grown to 5×10⁵ cells/mL, before filtered supernatants of bacterial cultures of LLC or LGG were added to the THP-1 cells at a dilution of 1:50 and incubated for 24 hours. Thereafter, the THP-1 cell suspensions were delivered to the ONF wound via a PEG-MAL hydrogel in a similar approach to the direct delivery of probiotics. Because THP-1 are a human cell line, an ONF wound model in NOD/SCID mice, which lack T and B cells and would not respond adversely to human macrophages was used. Strikingly, hydrogel-mediated delivery at day 0 and day 1 to the ONF of THP-1 cells that had been exposed to LLC for 24 hours significantly accelerated ONF healing, compared to controls of hydrogel alone, hydrogel containing THP-1 cells not exposed to bacteria, or hydrogel containing THP-1 cells exposed to LGG. Indeed, THP-1 cells that had been exposed to LLC induce the nearly complete healing of the ONF in 9 out of 10 (90%) of mice, compared to healing rates of between 50% and 75% in control group. Critically, only THP-1 cells exposed to LLC, but not to LGG induced healing, showing that the response is specific to the LLC probiotic, pointing to the notion that LLC secretes specific factors that induce phenotypic changes in macrophages favoring a pro-restitutive phenotype.

Experiments were performed to determine whether LLC directly impacts macrophage activity. In vitro cytokine analysis of human THP-1 macrophages stimulated with supernatant from LLC culture indicated an induced the production of anti-inflammatory cytokines, including IL-10 which is a potent anti-inflammatory cytokine, and IL-27, which induces differentiation of the diverse populations of T cells in the immune system, and upregulates IL-10.

What is claimed is:

1. A graft composition comprising a graft matrix comprising an exogenously added beneficial microbe or supernate or components therein and optionally macrophages and/or stem cells.

2. The graft composition of claim 1, wherein the beneficial microbe is Lactococcus lactis subsp. cremoris (LLC) ATCC® 19257.

3. The graft composition of claim 1, wherein the graft comprises macrophages.

4. The graft composition of claim 3, wherein the macrophages are pre-exposed to 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof.

5. The graft composition of claim 3, wherein the macrophages are pre-exposed to secretions of Lactococcus lactis subsp. cremoris (LLC) ATCC® 19257.

6. The graft composition of claim 1, wherein the graft is a polyethylene glycol-based hydrogel.

7. The graft composition of claim 6, wherein the polyethylene glycol-based hydrogel is polyethylene glycol maleimide (PEG-MAL) hydrogel.

8. A method of closing an oro-nasal fistula (ONF) comprising implanting an effective graft composition of claim 1 into the oro-nasal fistula.

9. The method of claim 8, wherein the beneficial microbe is Lactococcus lactis subsp. cremoris (LLC) ATCC® 19257.

10. The method of claim 8, wherein the graft comprises macrophages.

11. The method of claim 10, wherein the macrophages are pre-exposed to 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof.

12. The method of claim 10, wherein the macrophages are pre-exposed to secretions of Lactococcus lactis subsp. cremoris (LLC) ATCC® 19257.

13. The method of claim 8, wherein the graft is a polyethylene glycol (PEG) based hydrogel.

14. The method of claim 13, wherein the polyethylene glycol-based hydrogel is polyethylene glycol maleimide (PEG-MAL) hydrogel.

15. A graft composition comprising a graft matrix comprising macrophages and/or stem cells.

16. The graft composition of claim 15, wherein the macrophages and/or stem cells are pre-exposed to 2-amino-2-[2-(4-octyl-phenyl)-ethyl]-propane-1,3-diol (Fingolimod/FTY720), 2-amino-2-(hydroxymethyl)-4-(4-octylphenyl)butyl dihydrogen phosphate (Fingolimod phosphate/FTY720P), prodrug, or salt thereof.

17. The graft composition of claim 16, wherein the graft matrix is a hydrogel.

18. The graft composition of claim 17, wherein the hydrogel is a polyethylene glycol (PEG) based hydrogel.

19. The graft composition of claim 18, wherein the polyethylene glycol-based hydrogel is polyethylene glycol maleimide (PEG-MAL) hydrogel.

20. A method of closing an oro-nasal fistula (ONF) comprising implanting an effective graft composition of claim 16 into the oro-nasal fistula.

21. A kit comprising a graft matrix and optionally an exogenously added beneficial bacterium.


SEQUENCE LISTING (2 sequences)

SEQ ID NO: 1 - protein, 7 aa, synthetic construct
GRGDSPC

SEQ ID NO: 2 - protein, 16 aa, synthetic construct
GCRDGPQGIWGQDRCG